(19) United States
(12) Patent Application Publication
(10) Pub. No.: US 2011/0201836 A1
(43) Pub. Date: Aug. 18, 2011

(54) Process for extraction using silicones of differing partition coefficients

(76) Inventor: Anthony J. O'Lenick, JR., Dacula, GA (US)

(21) Appl. No.: 12/658,377

(22) Filed: Feb. 12, 2010

(51) Int. Cl. C07C 331/20

(52) U.S. Cl. 558/17

(57) ABSTRACT

The invention relates to a process for the extraction of actives from botanical materials using a series of silicone compounds having different partition coefficients. The botanical extracts are used in a wide range of applications, including in dermatocosmetic products. The selection of silicone-based products over solvents heretofore used allows for more efficient as well as more specific extraction of desired active-containing fractions from the plant material in a carrier that not only is more skin substantive but also enhances the retention of the dermatocosmetically-active constituents from botanical materials on the skin.

### FIELD OF THE INVENTION

The present invention relates to a process for the extraction of actives from botanical materials using a series of silicone compounds having different partition coefficients. The botanical extracts are used in a wide range of applications, including in dermatocosmetic products. The selection of silicone-based products over solvents heretofore used allows for more efficient as well as more specific extraction of desired active-containing fractions from the plant material in a carrier that not only is more skin substantive but also enhances the retention of the dermatocosmetically-active constituents from botanical materials on the skin.

### BACKGROUND OF THE INVENTION

Plants were the first and remain one of the most important sources of active compounds for medicinal and cosmetic products. Among the many skin-beneficial or “healing” properties associated with use of botanical extracts in topically-applied products are reduction in trans-epidermal water loss, improved barrier function, increased moisturizing, decreased inflammation and/or reddening, scavenging free radicals, protection from UV-induced photodamage. See, e.g., U.S. Pat. No. 6,861,077, the disclosures of which are incorporated herein by reference. The active ingredients, however, are generally present in low concentrations, often in different parts of the plant. The benefit(s) to be realized from using botanically-derived materials, particularly in the dermatopharmaceutical arts, therefore requires isolating and concentrating specific actives.

Over the centuries, a variety of techniques have been employed to extract active ingredients from botanicals. Solvent-based extraction is one such methodology. After segregating the plant material into its constituent parts (e.g., leaves, stems, fruits, branches, roots), the part with the desired active(s) is chosen, macerated (or similarly processed) and placed into the solvent. Materials that are soluble in the solvent are dissolved, leaving insoluble materials behind. Solvent-based extraction systems are recognized as having at least two important limitations: first, they are able to extract only those active materials that are soluble in the chosen solvent; second, because many solvents lack selectivity, compounds other than those that are desired are extracted. Not only can this dilute the purity of the desired active compound, it can also negatively impact the performance benefits of the extract.

A number of techniques are known to those of skill in the art for extracting water-soluble actives. For example, hot water extraction removes water-soluble materials, leaving behind oil-soluble materials as well as materials that are insoluble either in water or oil. Tinctures, produced by using hydro-alcoholic solutions, are another extraction vehicle well-known to skilled artisans. Inclusion of alcohol alters the polarity of water, producing different and marginally more efficient (e.g., higher-yield) extracts than water or alcohol alone. Propylene glycol is yet another commonly-used vehicle to extract water- and alcohol-soluble materials. Although generally recognized as safe by the FDA, certain consumer groups have raised health concerns regarding short and long-term health consequences about use of propylene glycol per se, attempting to implicate its use with conditions ranging from skin irritation and sensitization to potential reproductive and development toxicity.

In organic chemistry, extraction is a well-known technique for separating chemical constituents. It is a process by which a solute is extracted from a first solvent into a second solvent, where the two solvents are immiscible. One common extraction methodology used in organic chemistry involves combining in a separatory funnel water and diethyl ether. As is well-known to those of skill in the art, ether and water do not mix; without agitation, they rapidly separate into two phases or layers.

As used in the present application, the phrase “partition coefficient” is understood to mean the equilibrium distribution of the solute between the two immiscible solvent phases. When an organic compound is placed into a solvent mixture of ether and water, it separates, or partitions, into the water and ether phases. At equilibrium, the ratio of the concentrations of the organic solute in each of the solvent layers is its partition coefficient. As is well-known to those of skill in the art, extracting a compound with an ether/water partition coefficient of 80 will result in eighty percent extraction of the material during each separation procedure.

It is also well-known to those of skill in the art that extraction procedures of the type described above are, by their very nature, inefficient, e.g. in terms of maximizing the yield of a desired organic component. In the example of a 1,000 mg sample of botanical material having an ether/water partition coefficient of 80, after a first pass in a separatory funnel, 800 mg of the botanical will be extracted into the ether phase. In order to recover still more botanical material from the remaining 200 mg (e.g., in the water phase), the separation procedure is repeated, each time with fresh ether solvent. The second pass, for example, is expected remove 140 additional mg, or 80% of the ether-soluble botanical components. However, even with multiple repetitions, some percentage of botanical extract eludes extraction. Thus, there has been and remains a long-felt need to more efficiently and specifically extract actives from botanical materials.

In biochemical applications, partition coefficients is often expressed in terms of an octanol/water coefficient (K_{o/w})—the ratio at equilibrium of the concentration of a non-ionized organic compound in an organic solvent (e.g., octanol) versus the concentration of the compound water. K_{o/w} has been correlated with lipophilicity, the affinity of a compound for a lipophilic environment. Compounds having K_{o/w} of greater than one are lipophilic; those below one are hydrophilic. K_{o/w} thus provides an important predictive measure of the ability of a compound to pass through the acid mantle and permeate the lipophilic membranes of cells of the epidermis and dermis Polar (i.e., hydrophilic) materials extract a different, but also highly-desirable class of compounds from botanical materials. The ability to extract a variety actives (i.e., both lipophilic and hydrophilic) over a wide range of polarities has heretofore been unattainable.

Prior to the present invention, extraction technology has been limited by the nature of available solvents (e.g., oil, aqueous, hydroalcohol and glycol). Surprisingly, applicants have discovered that a series of silicone molecules customized to alter their solubilities in oil, water, silicone and fluoro-solvents produces an almost infinite number of extraction solvents that selectively and specifically extract desired active fractions from botanical materials. In addition to improving extraction efficiency, the customized silicone polymer extraction vehicles of the present invention contribute to the aesthetics and functionality of topical formulations—they impart outstanding skin feel and are non-irritating while retaining the actives on the skin for longer periods of time.

The present invention is thus directed to a process for the extraction of compounds from botanical compositions (plant material) which comprises contacting the botanical material with an effective extraction concentration of a silicone polymer that has been engineered to have differing amounts of silicone-soluble, oil-soluble, fluoro-soluble and water-soluble groups in the backbone of the silicone molecule. By altering the ratio of each such group relative to others, a series of extracts covering a wide range of partition coefficients can be created, thereby effectively extracting materials from plants. Thus, as used in the present application, the term extraction is understood to mean a process by which botanical materials are extracted from one or more plant parts into a silicone polymer having differing solubilities in oil, water, silicone and fluoro-solvents.

The silicone polymers based on partition coefficient technology of the present invention allow for customizing a menstruum to extract not only more materials but also narrower ranges of materials than heretofore has been possible. This specificity offers a number of advantages over the prior art: First, by varying the ratio of silicone-soluble to oil-soluble to water-soluble to fluoro-soluble portions in the molecule (and in so doing creating a series of polymeric materials of differing partition coefficients), many different fractions can be extracted from the same starting plant material. Second, the resulting extracts can each be evaluated for their effects on reducing the signs of biological and/or or photoaging (e.g., appearance of fine lines and wrinkles) as well as analgesic, anti-inflammatory, free radical scavenging, anti-microbial and other desired functional. Based on a “range-finding” series of silicone polymer extraction vehicles, further refinements can be made to the polymers to maximize the concentration of and/or further isolate compounds of interest. Indeed, many compounds extracted by using the processes of the present invention may have been unavailable. Yet another advantage of the present invention is in terms of environmental stewardship; because extraction of desired actives is more efficient, smaller quantities of plant materials need to be harvested.

### SUMMARY OF THE INVENTION

The present invention is directed to a process for more efficiently and more specifically extracting dermatocosmetically-active compounds from plant parts based on polymeric silicone extraction vehicles with segments of differing partition coefficients. By altering the ratios of mutually-insoluble groups—silicone-soluble, oil-soluble, water-soluble, and fluoro-soluble—on a silicone polymer backbone, actives of differing polarities can be extracted in greater quantities with higher specificity. Constituents in the resulting botanical extracts may have antioxidant, anti-inflammatory and/or anti-microbial properties.

### DETAILED DESCRIPTION OF THE INVENTION

The present invention is directed to a process for extracting compounds from plants which comprises contacting the plant material with an effective extraction concentration of a silicone compound conforming to the following structure;

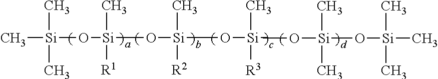

wherein;
R¹ is —(CH₂CH₂O)ₑ—(CH₂CH(CH₃)O)_{f}—(CH₂CH₂O)_{g}H;
R² is —(CH₂)ₕ—CH₃;
R³ is —(CH₂)₂—(CF₂)ᵢCF₃;

a is an integer ranging from 0 to 20;

b is an integer ranging from 0 to 20;

c is n integer ranging from 0 to 20;

d is an integer ranging from 0 to 200;

e is an integer ranging from 0 to 20;

f is an integer ranging from 0 to 20;

g is an integer ranging from 0 to 20;

h is an integer ranging from 0 to 35;

i is an integer ranging from 0 to 9.

It will be clearly understood that if any one value of a, b, c, or d is non-zero and the others are all zero, the resulting silicone material will have solubility in the non-zero phase and consequently extract materials with an affinity for that phase.

According to one aspect of the present invention, “a” is a non-zero value and “b”, “c”, and “d” are all zero. The resulting silicone material will be polar and will extract polar components from the plant material.

According to another aspect of the present invention, “b” is a non-zero value and “a”, “c” and “d” are all zero. In this embodiment, the silicone-material will be oil-soluble and, consequently, will extract oil-soluble constituents from the plant material.

In another aspect of the present invention, where “c” is non-zero and “a”, “b” and “d” are all zero, fluoro-soluble constituents in the plant materials will be extracted.

In yet another aspect of the present invention, where “d” is non-zero, and “a”, “b”, and “c” are all zero, silicone-soluble materials will be extracted.

In a preferred embodiment of the present invention, more than one of “a”, “b”, “c” or “d” are non-zero values.

In a most preferred embodiment of the present invention, each of “a”, “b”, “c” or “d” is a non-zero value. Because molecules according to this most preferred embodiment have varying amounts of multiple groups, each with their own solubility, they maximize not only the yield but also the specificity of desired actives.

Raw Materials

Silicone Menstruum Compounds

Silicone compounds suitable for use in practicing the extraction method of the present invention are commercially available from a variety of sources, including Siltech LLC (Dacula, Ga.) which conform to the following structure:

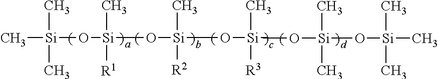

wherein;
R¹ is —(CH₂CH₂O)ₑ—(CH₂CH(CH₃)O)_{f}—(CH₂CH₂O)_{g}H;
R² is —(CH₂)ₕ—CH₃;
R³ is —(CH₂)₂—(CF₂)ᵢCF₃;

a is an integer ranging from 0 to 20;

b is an integer ranging from 0 to 20;

c is n integer ranging from 0 to 20;

d is an integer ranging from 0 to 200;

e is an integer ranging from 0 to 20;

f is an integer ranging from 0 to 20;

g is an integer ranging from 0 to 20;

h is an integer ranging from 0 to 36;

i is an integer ranging from 0 to 9.

The values given in the following examples are determined by ²⁹Si-NMR and ¹³C-NMR, using methodologies known to those skilled in the art.

| Example | a | b | c | d | e | f | g | h | i |
|---|---|---|---|---|---|---|---|---|---|
| 1 | 1 | 0 | 0 | 0 | 5 | 1 | 5 | 0 | 0 |
| 2 | 4 | 0 | 0 | 10 | 0 | 0 | 10 | 0 | 0 |
| 3 | 8 | 1 | 0 | 10 | 0 | 0 | 20 | 6 | 0 |
| 4 | 10 | 0 | 20 | 10 | 20 | 20 | 20 | 11 | 8 |
| 5 | 20 | 20 | 10 | 200 | 10 | 5 | 10 | 35 | 8 |
| 6 | 0 | 0 | 20 | 20 | 0 | 0 | 0 | 17 | 1 |
| 7 | 0 | 10 | 0 | 0 | 0 | 0 | 0 | 8 | 0 |
| 8 | 0 | 0 | 20 | 0 | 0 | 0 | 0 | 0 | 8 |
| 9 | 20 | 20 | 20 | 200 | 20 | 20 | 20 | 35 | 8 |
| 10 | 0 | 2 | 0 | 4 | 0 | 0 | 0 | 15 | 0 |
| 11 | 0 | 0 | 0 | 10 | 0 | 0 | 0 | 0 | 0 |
| 12 | 10 | 0 | 0 | 20 | 0 | 0 | 12 | 0 | 0 |
| 13 | 8 | 2 | 1 | 20 | 0 | 5 | 10 | 11 | 8 |
| 14 | 6 | 4 | 0 | 20 | 0 | 0 | 10 | 11 | 0 |
| 15 | 4 | 6 | 2 | 20 | 10 | 5 | 10 | 15 | 8 |
| 16 | 2 | 8 | 0 | 20 | 2 | 2 | 6 | 15 | 3 |
| 17 | 0 | 0 | 4 | 8 | 0 | 0 | 0 | 0 | 8 |
| 18 | 5 | 0 | 5 | 200 | 7 | 5 | 3 | 35 | 8 |

Plant Materials

The process of the present invention is broadly applicable to a wide variety of plant materials, including but not limited to roots, leaves, bark and berries. As applicable and necessary, the materials chosen for extraction are dried and ground. Next, they are placed into a filter bag over which warm silicone-based menstruum is passed for a period of from between 12 and 24 hours. The progress of the extraction is monitored by FTIR. As the extraction proceeds, new infrared bands develop, indicating that materials are being extracted. When a “constant” spectra emerges, one in which the bands change to a minor extent, if at all, the extraction is considered to be complete.

Plant Examples

| Example | Plant |
|---|---|
| 19 | Aloe Vera |
| 20 | Arbutus |
| 21 | Arnica |
| 22 | Borage |
| 23 | Broccoli sprout powder |
| 24 | Capsicum |
| 25 | Chamomile |
| 26 | Gentian |
| 27 | Ginkgo |
| 28 | Ginseng |
| 29 | Goldenseal |
| 30 | Pomegranate |
| 31 | Raspberry |
| 32 | Rose Hips |
| 33 | Rosemary |
| 34 | Sage |
| 35 | St. John's Wort |
| 36 | Valerian |
| 37 | Wintergreen |
| 38 | Yellow Dock |

The above-listed plant materials are available from a variety sources, including plant nurseries, ethnic and organic groceries and health food stores. Broccoli sprouts (Example 22) are available from Natural Sprouts, LLC (Springfield, Mo.).

Extraction General Procedure

Dried and crushed plant materials (examples 19-38) are placed in a filter bag having a pore size of 100 microns. The amount of plant materials is from about 1% to about 10% by weight, meaning the silicone-based menstruum comprises from about 9% to 99% of the weight. The selected menstruum is added to a recirculaton vessel, heated to between 60° C. and 90° C., and recirculated through the filter bag for a period of 12 and 24 hours. The progress of the extract is monitored by FTIR as described above.

The procedure as set described immediately above is followed in Examples 39-62. 10 grams of plant material are added to filter bag. 90 grams of menstruum, heated to 90° C., is recirculated through the filter for 24 hours. The resulting materials are used without purification.

| | Plant | Menstruum |
|---|---|---|
| Example | Example | Example |
| 39 | 19 | 1 |
| 40 | 20 | 2 |
| 41 | 21 | 3 |
| 42 | 22 | 4 |
| 43 | 23 | 5 |
| 44 | 24 | 6 |
| 45 | 25 | 7 |
| 46 | 26 | 8 |
| 47 | 27 | 9 |
| 48 | 28 | 10 |
| 49 | 29 | 11 |
| 50 | 30 | 12 |
| 51 | 31 | 13 |
| 52 | 32 | 14 |
| 53 | 33 | 15 |
| 54 | 34 | 16 |
| 55 | 35 | 17 |
| 56 | 18 | 10 |
| 57 | 18 | 11 |
| 58 | 18 | 12 |
| 59 | 18 | 17 |
| 60 | 36 | 12 |
| 61 | 37 | 12 |
| 62 | 38 | 12 |

Applications Examples

The improved extraction efficiencies achieved by using silicone polymer menstrua of the present invention are further illustrated based on extracts of broccoli sprouts, a class of materials that have been reported by researchers at John Hopkins University to be effective in protecting against ultraviolet radiation induced skin damage, including cancer. One constituent of particular interest in broccoli sprouts is sulforaphane (C₆H₁₁NOS₂), a breakdown product of glucosinolate glucoraphanin. Sulforaphane is also an isothiocyanate, more particularly 4-methylsulfinylbutyl isothiocyanate and (−)-1-isothiocyanato-4(R)-(methylsulfinyl)butane. As discussed below, four silicone polymer menstrua extract four different active fractions with four different FTIR spectra. Spectral subtraction of the menstrua allows identification of groups present in the extracted materials. Menstrua based on partition coefficient technology of the present invention allow for the rapid screening and effective extraction of this class of materials from broccoli sprouts as well as other cruciferous materials.

### Example 56

The silicone menstruum of Example 56 is rich in alkyl-soluble groups an extracts 3% of actives. Based on FTIR spectral subtraction are ester, unsaturation, some amine groups and some ketones. The extract changes in color from water white to pale yellow. The skin feel of the product is quite cosmetically elegant, providing a smooth feel with outstanding glide and spread. The extract of this example can be used as is (i.e., in the menstruum without further modification) as well as in a topical emulsion system by methods known to those in the art.

### Example 57

This menstruum, rich in silicone-soluble groups, extractes 0.9% of the plant material. The functional groups present on the FTIR after spectral subtraction are primarily alcohol and ketones. The extract has no color change. The skin feel is cosmetically appealing, providing a dry powdery feel with outstanding spread. The extract can be used as is or put into a topical emulsion systems using methods known to those skilled in the art. This fraction, unlike the starting material, has a distinct UV spectra. The extract of this example can be used as is (i.e., in the menstruum without further modification) as well as in a topical emulsion system by methods known to those in the art.

### Example 58

This menstruum, rich in water-soluble groups, is most effective of the four examples in extracting material; 7% by weight is extracted. The functional groups present on the FTIR after spectral subtraction are alcohols, some esters, a large amount of unsaturation, and some ketones. The extract changes in color from water white to intensely yellow. The skin feel of the product is cosmetically appealing, providing a smooth feel with outstanding glide and spread. The extract can be used as is or put into water. This extract has a noticeable sulfur smell, which may be an indication of sulforaphane being present.

### Example 59

This menstruum, rich in fluoro-soluble groups, extracts 1.2% of the plant material. The functional groups present on the FTIR after spectral subtraction are ester, and unsaturation. The extract changes in color from water white to pale yellow. The skin feel of the product is quite cosmetically appealing, providing a smooth feel with outstanding spread and waterproofing properties.

The International Cosmetic Ingredient Dictionary and Handbook published by the Cosmetic, Toiletries & Fragrance Association describes a wide variety of non-limiting dermatocosmetic ingredients that can be used in combination with extracts achieved by the method of the present invention. Non-limiting examples of these ingredients include emulsifiers, surfactants, thickeners, anti-oxidants, anti-inflammatory agents, sunscreen actives, preservatives, natural moisturizing factors, emollients, humectants, moisturizers and film formers (e.g., polymers for aiding the film-forming properties and substantivity of the composition). Further examples of additional ingredients which are suitable for use in combination with the extracts according to the present invention are disclosed in U.S. Pat. Nos. 6,492,326 and 6,277,892, the disclosures of which are incorporated herein by reference.

While the illustrative embodiments of the invention have been described with particularity, it will be understood that various other modifications will be apparent to, and can be readily made by, those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

We claim:

1. A process for extracting materials from plants which comprises contacting the plant material with an effective extraction concentration of a menstruum conforming to the following structure: 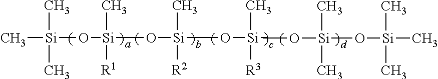
wherein;
R¹ is —(CH₂CH₂O)ₑ—(CH₂CH(CH₃)O)_{f}—(CH₂CH₂O)_{g}H;
R² is —(CH₂)ₕ—CH₃;
R³ is —(CH₂)₂—(CF₂)ᵢCF₃;
a is an integer ranging from 0 to 20;
b is an integer ranging from 0 to 20;
c is n integer ranging from 0 to 20;
d is an integer ranging from 0 to 200;
e is an integer ranging from 0 to 20;
f is an integer ranging from 0 to 20;
g is an integer ranging from 0 to 20;
h is an integer ranging from 0 to 36;
i is an integer ranging from 0 to 9.

2. A process of claim 1 wherein one of a, b, c and d is a non-zero.

3. A process of claim 1 wherein two of a, b, c and d are non-zero integers.

4. A process of claim 1 wherein three of a, b, c and d are non-zero integers.

5. A process of claim 1 wherein each of a, b, c and d are non-zero integers.

6. A dermatocosmetic composition comprising plant materials extracted using the process of claim 1.

7. A dermatocosmetic composition comprising plant materials extracted using the process of claim 2.

8. A dermatocosmetic composition comprising plant materials extracted using the process of claim 3.

9. A dermatocosmetic composition comprising plant materials extracted using the process of claim 4.

10. A dermatocosmetic composition comprising plant materials extracted using the process of claim 5.
